(19) United States
(12) Patent Application Publication
(10) Pub. No.: US 2025/0152869 A1
(43) Pub. Date: May 15, 2025

(54) LUNG DEPOSITION ANALYSIS

(71) Applicant: Pulmotree Medical GmbH, München (DE)

(72) Inventors: Ulf Krüger, München (DE); Pascal Förner, Wallenfels (DE)

(21) Appl. No.: 18/833,264

(22) PCT Filed: Jan. 26, 2023
(86) PCT No.: PCT/EP2023/051882

§ 371 Date: Sep. 16, 2024

(30) Foreign Application Priority Data
Jan. 26, 2022 (EP) 22153537.0

(51) Int. Cl. A61M 15/00

(52) U.S. Cl. CPC A61M 15/009; A61M 2202/064; A61M 2205/3334; A61M 2209/02

(57) ABSTRACT

Disclosed is an analysis system for determining the deposition of an aerosol for a breathing apparatus with an analysis device comprising an air duct, a first air sensor, a control unit and an application component, wherein the air duct is set up to guide air along the first air sensor to the application component and vice versa, the first air sensor is set up to detect at least a first air flow parameter of the air guided along the first air sensor, and the control unit is set up to control the first air sensor in such a way that a time characteristic of the first air flow parameter can be measured as a first measurement result, and the first measurement result can be transmitted to an assessment device.

### FIELD OF THE INVENTION

The invention relates to an analysis system for determining the deposition of an aerosol for a breathing apparatus, a method for determining the deposition of an aerosol for a breathing apparatus and a use of such an analysis system for carrying out such a method.

### STATE OF THE ART

Medical devices are available that can be used to introduce medication into a patient's respiratory system. Such medical devices include inhalers, which in turn are divided into metered dose inhalers (pressurized gas or normal pressure), powder inhalers, soft mist inhalers or soft inhalers and nebulizers.

Nebulizers are used to separate fine droplets of liquid from a liquid medication reservoir. This creates an aerosol that can be inhaled by the patient through a mouthpiece. Depending on the functional principle used, nebulizers are divided into nozzle nebulizers, ultrasonic nebulizers or oscillating membrane nebulizers (mesh nebulizers).

In general, these medical devices are used to add aerosols of medication to a stream of air taken in by the patient. The drug aerosols then enter the patient's respiratory system via the patient's body orifices, for example their mouth or nose, where they are then at least partially deposited.

The location of the deposition or deposition of medication or medication aerosols in the respiratory system, for example in the lungs, depends on different variables, which include, for example, the aerosol characteristics (diameter or particle size and diameter distribution or particle size distribution), the inhalation speed or inhalation flow (volume flow) and the inhaled volume. A change in these variables influences the deposition location of the aerosol. For example, the aerosol can be deposited in the lung periphery, bronchial or pharyngeal region. However, as each drug is intended for a specific site of action or deposition or can develop its effect in the best possible way at this site, it is important to reach this site of action in the best possible way or with the greatest possible efficiency. The deposition site of drug aerosols in the respiratory system can currently only be determined by clinical deposition studies. These are based on imaging procedures, such as X-ray diagnostics or scintigraphy, and are therefore very time-consuming. In contrast, models exist for calculating the deposition site of drug aerosols in the respiratory system, although these are based on standardized flow curves, assumptions about aerosol characteristics and results from clinical deposition studies, which means that only a theoretical approximation can be made. For example, no account is taken of how the patient actually inhaled during the respective inhalation.

Accordingly, the determination of the deposition of drug aerosols in the respiratory system is either very complex or very inaccurate.

### DESCRIPTION OF THE INVENTION

An object of the present invention is therefore to provide a system and a method with which simple and accurate dosing of drug aerosols is made possible.

This purpose is addressed by an analysis system according to claim 1, a method according to claim 8 and a use of an analysis system for carrying out a method according to claim 15. Further features of the invention are contained in the subclaims.

An analysis system according to the invention for determining the deposition of an aerosol for a breathing apparatus has an analysis device comprising an air duct, a first air sensor, a control unit and an application component, wherein the air duct is arranged to guide air along the first air sensor to the application component and vice versa, the first air sensor is set up to detect at least a first air flow parameter of the air duct along the first air sensor, and the control unit is set up to control the first air sensor in such a way that a time characteristic of the first air flow parameter can be measured as a first measurement result, and the first measurement result can be transmitted to an assessment device. This enables a simple and precise determination of the deposition of an aerosol for a breathing apparatus, i.e. conclusions as to the extent to which, to what extent and at what location aerosols were deposited in the breathing apparatus via the application component during an application of the analysis system according to the invention. This in turn enables precise dosing of drug aerosols. The breathing apparatus is a component of an organism with which air from the environment can be absorbed and air from the same can be released into the environment. The assessment device is suitable for receiving the first measurement result. An additional receiving component can be provided for this purpose.

Preferably, the analysis system has the assessment device, and the assessment device is set up to carry out a deposition determination using the first measurement result, at least one deposition parameter and a deposition model, and to output a deposition result as the result of the deposition determination. This makes it possible to model pharmacokinetic processes, enabling more targeted and effective treatment. The deposition result shows to what extent and at what position in the respiratory system drug aerosols have been deposited or deposited. The deposition result can then be used to draw conclusions about how the drug aerosols can be absorbed.

Preferably, the assessment device is set up to carry out the deposition determination independently of a network connection. This enables better or simplified protection of patient data.

Preferably, the control unit is also set up to store the first measurement result and the first measurement result can be transmitted from the control unit to the assessment device. This makes it possible to carry out the deposition determination after a completed measurement, which enables a robust and independent assessment.

Preferably, the control unit is also set up to control the first air sensor in such a way that a large number of successive partial measurement results can be measured. The partial measurement results can be transmitted individually and bundled to the assessment device. This makes it possible to carry out the deposition determination during a measurement or series of measurements and to constantly update the deposition result, whereby application information can be made available to the patient immediately. This also makes it possible to temporarily pause the delivery of drug aerosols, for example if a partial measurement result indicates that the deposition of the drug aerosol would not be as desired or potentially even harmful. This also improves patient protection.

Preferably, the analysis system also has a second air sensor, which is arranged in the air duct, and the control unit is also set up to control the second air sensor in such a way that a time profile of the second air flow parameter can be measured as a second measurement result. The second measurement result can be transmitted to the assessment device, and the assessment device is also set up to additionally perform the deposition determination using the second measurement result. It is also possible for the analysis system to have a third or a plurality of air sensors so that a third or a plurality of measurement results can be measured. This makes it possible to carry out a more precise deposition determination.

Preferably, the first air sensor is also set up to detect a second air flow parameter of the air passing along the first air sensor, and the control unit is also set up to control the first air sensor in such a way that a time course of the second air flow parameter can be measured as a second measurement result. The second measurement result can be transmitted to the assessment device, and the assessment device is also set up to additionally carry out the deposition determination using the second measurement result. This makes it possible to carry out a more precise deposition determination.

Preferably, the analysis system also has an inhaler, whereby the inhaler is in particular a nebulizer, particularly preferably a mesh nebulizer. In this way, it is possible to carry out a deposition determination in the course of an inhalation of, for example, drug aerosols by a patient without additional devices, which simplifies handling.

A method according to the invention for determining the deposition of an aerosol for a breathing apparatus comprises the steps of (i.) guiding air in an air duct along a first air sensor to an application component, (ii.) detecting at least a first air flow parameter of the air ducted along the first air sensor by means of the first air sensor, (iii.) controlling the first air sensor by means of a control unit such that a time course of the first air flow parameter is measured as a first measurement result, (iv.) performing a deposition determination using the first measurement result, at least one deposition parameter and a deposition model, and (v.) outputting a deposition result as a result of the deposition determination performed during step iv. The deposition determination relates to an area or areas and the amount of aerosols deposited at the area or areas. This enables a simple and accurate determination of the deposition of an aerosol for a breathing apparatus, which in turn enables accurate dosing of drug aerosols.

Furthermore, the deposition result can be used to draw conclusions as to whether the targeted or desired deposition site matches the actual deposition site, i.e. the one that is output via the deposition result. In this way, additional conclusions can be drawn regarding deviations or characteristics of vital parameters or patient-specific parameters, for example the blood plasma drug concentration.

Preferably, the aerosols are drug aerosols that can be administered to a patient via their respiratory system. In this way, it is easy to draw conclusions about any conditions that may favor therapy.

Preferably, between steps iii. and iv. the first measurement result is received by means of an assessment device and step iv. is carried out by means of the assessment device. The aspects described above with regard to the assessment device apply analogously here.

Preferably, during step iii. the first measurement result after the measurement is stored in the control unit and then transmitted from the control unit to the assessment device. This makes it possible to carry out the deposition determination after a completed measurement, which enables a robust and independent assessment.

Preferably, during step iii. the first air sensor is controlled by the control unit such that a plurality of successive partial measurement results are measured. The aspects described above with regard to the partial measurement results apply analogously here.

Preferably, a deposition determination is carried out during step iv. at a plurality of successive points in time, in each case with the partial measurement results measured up to the current point in time, and a current deposition result is output during step v. at the plurality of successive points in time, in each case with the partial measurement results measured up to the current point in time during step iii. The aspects described above with regard to the possibility of performing the deposition determination during a measurement or series of measurements as well as feedback to the patient during an inhalation apply analogously here. The partial measurement results measured up to the current point in time are or may have been transmitted to the assessment device.

Preferably, during step i. air is also guided along a second air sensor to the application component, during step ii. a second air flow parameter of the air ducted along the second air sensor is also detected by means of the second air sensor, during step iii. the second air sensor is also controlled by means of the control unit in such a way that a time course of the second air flow parameter is measured as a second measurement result, and during step iv. the deposition determination is also carried out using the second measurement result. The aspects described above in relation to the second air sensor and the second measurement result apply analogously here. If necessary, the second measurement result is transmitted to the assessment device and the second measurement result is also received between steps iii. and iv. by means of the assessment device.

Preferably, during step ii. a second air flow parameter of the air ducted along the first air sensor is furthermore detected by means of the first air sensor, during step iii. the first air sensor is furthermore controlled by means of the control unit such that a time course of the second air flow parameter is measured as a second measurement result, and during step iv. the deposition determination is additionally carried out on the basis of the second measurement result. The aspects described above with regard to the suitability of the first air sensor to detect a second or third air flow parameter or a plurality of air flow parameters apply analogously here. If necessary, at the end of step ii. the second measurement result is transmitted to the assessment device, and between steps iii. and iv. the second measurement result is also received by means of the assessment device.

A use of an analysis system according to the invention for carrying out a method according to the invention takes place in particular for a human lung. This enables a simple and accurate determination of the deposition of an aerosol for a breathing apparatus, which in turn enables an accurate dosing of drug aerosols.

### BRIEF DESCRIPTION OF THE FIGURES

FIG. 1a shows an abstract representation of a preferred analysis system with a first air sensor.

FIG. 1b shows an abstract representation of a preferred analysis system with a first air sensor and a second air sensor.

FIG. 1c shows an abstract representation of the analysis system shown in FIG. 1a with an inhaler.

FIG. 2 shows an abstract representation of the analysis system shown in FIG. 1c with a breathing apparatus.

FIG. 3 shows a preferred method.

FIG. 4a shows a preferred deposition result.

FIG. 4b shows another preferred deposition result.

FIG. 5 shows a time course of a first air flow parameter as a preferred first measurement result.

FIG. 6a shows a head unit with application component in a side sectional view with the air flow drawn in.

FIG. 6b shows the head unit shown in FIG. 6a in a sectional view from below.

### DESCRIPTION OF THE PREFERRED EMBODIMENTS

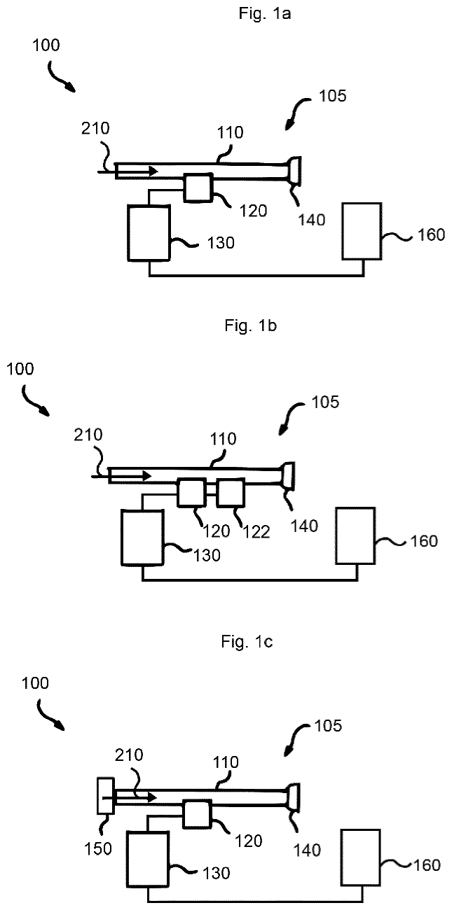

FIG. 1a shows an abstract representation of a preferred analysis system 100 with a first air sensor 120. The analysis system 100 comprises an analysis device 105. The analysis device 105 has an air duct 110. Air 210 can be guided through the air duct 110. In the embodiment shown in FIG. 1a, the air 210 is guided from the left end to an application component 140 arranged at the right end. However, the air 210 can also be guided in the opposite direction. The first air sensor 120 is arranged in such a way that the air 210 guided in the air duct 110 is guided along the first air sensor 120. A control unit 130 is connected to the first air sensor 120. In turn, an assessment device 160 is connected to the control unit 130. The connection between the control unit 130 and the first air sensor 120 can be wired, as shown, but can also be provided wirelessly, so that wired or wireless signal transmission is possible. Furthermore, the first air sensor 120 and the control unit 130 can be combined in one component. Furthermore, the application component 140 and the air duct 110 can be designed as a single-piece component.

The first air sensor 120 is, for example, an air pressure sensor, so that the air pressure can be measured as the first air flow parameter, an air mass sensor, so that the air mass can be measured as the first air flow parameter, an air volume sensor, so that the air volume or the inhalation flow can be measured as the first air flow parameter, or an air velocity sensor, so that the air velocity or the inhalation velocity can be measured as the first air flow parameter. Other types of air sensor are also possible, with which further air flow parameters can be measured, for example air temperature and humidity. Such additional air flow parameters include not only fluid mechanical parameters, but also general parameters relating to the air of the air flow.

By measuring the inhalation velocity or the inhalation flow as an air flow parameter, a continuous calculation of the volume flow is possible. In addition to carrying out a deposition analysis, further actions can be initiated based on or with the help of the air flow parameters. For example, if the volume flow exceeds or falls below certain limit values, certain actions of the analysis system 100 can be triggered automatically. For example, a corresponding control signal can be generated which ensures that an aerosol generator connected to the analysis system 100 is switched on or off or paused. Furthermore, a corresponding control signal can be generated which ensures that the user of, for example, an inhaler 150 (see FIG. 1c) connected to the analysis system receives feedback on the usage behavior. The feedback can be haptic, visual or acoustic and can also be app-based in conjunction with a mobile device.

Other possible airflow parameters include inspiratory volume (IV), peak inspiratory flow (PIF) and airway resistance (RAW).

The airway 110 may be a straight tube in the simplest case, but may also be a curved tube, a variable diameter tube, a complex branched system, or any other system that can be used to direct air in a preferred manner. In particular, the air duct 110 can be designed such that the air is guided along the first air sensor 120 as a secondary flow or in a secondary arm. This has the advantage that the risk of contamination can be reduced. If the air is guided as a bypass flow, a bypass flow measurement principle can be used so that the characteristics of the main flow relevant for calculating the deposition location are taken into account instead of the bypass flow when a deposition determination is carried out using the first measurement result.

A deposition model can be stored in the analysis system 100, for example in the assessment device 160. The deposition model can be stored algorithmically. The deposition model can have empirical elements and elements based on mathematical or physical models, be completely empirically based or be completely based on mathematical or physical models.

One possible deposition model is, for example, the “Human Respiratory Tract Model for Radiological Protection” of the International Commission on Radiological Protection (“ICRP 66”). There are other deposition models of a commercial nature, some of which are significantly more accurate than the ICRP 66 deposition model. For example, there are commercial deposition models that are specifically designed for pharmaceutical aerosols.

FIG. 1b shows an abstract representation of a preferred analysis system 100 with a first air sensor 120 and a second air sensor 122. The analysis system 100 shown in FIG. 1b differs from the analysis system 100 shown in FIG. 1a in that it has a second air sensor 122. The second air sensor 122 is connected to the control unit 130 via the first air sensor 120 and to the assessment device 160 via the control unit 130. The second air sensor 122 can also be connected directly to the control unit 130.

The analysis device 105 may further comprise further sensors, in particular air temperature and humidity sensors.

As shown, the assessment device 160 can be provided as a physical component of the analysis system 100, but can also be provided separately from the latter, for example as being outsourced to a cloud.

The assessment device 160 can also be provided in several parts, for example in two parts, with each part of the assessment device 160 performing a part of the deposition determination. The deposition result can also be output to different terminal devices.

By means of the first air sensor 120 and the second air sensor 122, it is possible, for example, to determine the air pressure at different positions or at different times, so that a time-dependent or location-dependent differential pressure can be used for the deposition determination. It is also possible, for example, to measure both air pressure and air velocity or any other possible combination of different air flow parameters. A higher number of air sensors 120, 122 tends to achieve a higher measurement accuracy.

It is also possible that the first air sensor 120 is set up to detect a third or a plurality of air flow parameters, so that a third or a plurality of measurement results can be measured. It is also possible that a combination of several air sensors 120, 122 is used, each of which is set up to detect one or more air flow parameters.

FIG. 1c shows an abstract representation of the analysis system 100 shown in FIG. 1a with an inhaler 150. The inhaler 150 is arranged on the left side of the air duct 110. In the arrangement shown, air 210 originating from the inhaler 150 can be guided through the air duct 110, whereby the air 210 in the inhaler 150 can be enriched with aerosols, in particular with drug aerosols.

The inhaler 150 can, for example, be a metered dose inhaler (MDI), a powder inhaler, a soft mist inhaler or soft inhaler or a nebulizer. The nebulizer can be a jet nebulizer, an ultrasonic nebulizer or a vibrating membrane nebulizer (mesh nebulizer).

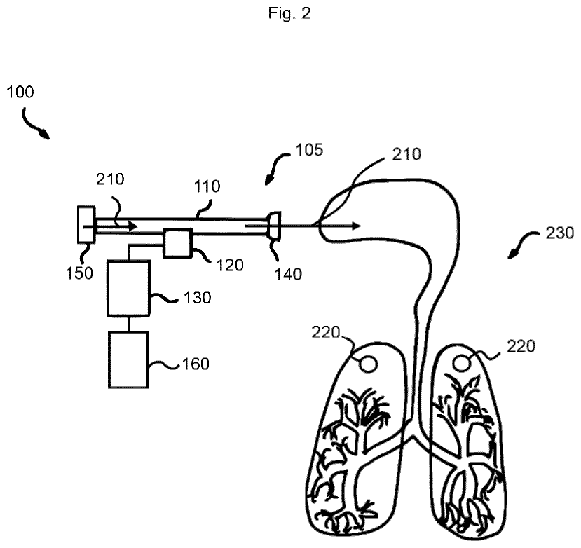

FIG. 2 shows an abstract representation of the analysis system 100 shown in FIG. 1c with a breathing apparatus 230. The analysis system 100 is arranged in relation to the breathing apparatus 230 in such a way that the air 210 can be fed into the breathing apparatus 230 via the application component 140. The breathing apparatus 230 shown is a human breathing apparatus. Aerosols 220 are arranged or deposited in the center left and right of the breathing apparatus 230. The aerosol 220 originates from the inhaler 150, but can also originate from a respirator, i.e. be applied orally. In the case of oral application by means of a respirator, the aerosol 220 is generated directly in the respirator. The analysis system 100 can then be integrated directly into the ventilator. The aerosol 220 can also be applied nasally. Accordingly, the application component 140 can, for example, be a mouthpiece or a breathing mask for the inhaler 150, but also a nasal attachment for nasal application or a breathing mask for a ventilator.

If the aerosol 220 comes from a ventilator, measurement results based on other air flow parameters measured in the ventilator can also be transmitted to the assessment device 160. In this way, the breathing cycle specified by the ventilator or the ventilation parameters set on the ventilator can also be taken into account in the assessment. It is also possible to use an additional device for controlled inhalation, from which parameters of the breathing cycle or set inhalation parameters can then be additionally taken into account in the assessment in the same way as the ventilator. The same is conceivable for additional devices for controlled nasal application.

The breathing apparatus 230 can be a human breathing apparatus and comprise the nose, the paranasal sinuses, the pharynx, the larynx, the trachea, the left and right main tracheal branches, the main bronchus (bronchus principalis), the bronchi, the bronchioles, the alveolar ducts and the alveoli.

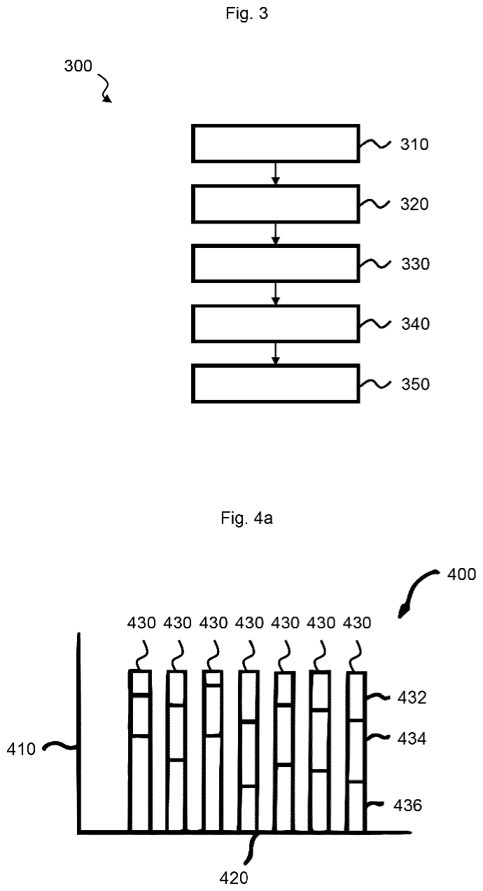

FIG. 3 shows a preferred method 300. The method 300 starts with a step 310 during which air is guided in an air duct along a first air sensor to an application component. This is followed by a step 320, during which at least a first air flow parameter of the air ducted along the first air sensor is detected by means of the first air sensor. This is followed by a step 330, during which the first air sensor is controlled by means of a control unit in such a way that a time characteristic of the first air flow parameter is measured as a first measurement result. This is followed by a step 340, during which a deposition determination is carried out using the first measurement result, at least one deposition parameter and a deposition model. The method 300 shown in FIG. 3 ends with a step 350, during which a deposition result is output as a result of the deposition determination performed during step 340.

The at least one deposition parameter can be patient-specific or aerosol-specific. Patient-specific deposition parameters may be, for example, the age, gender, ethnicity, weight and medical indication of the patient. Aerosol-specific deposition parameters can be the size distribution of the aerosol particles, the chemical composition of the aerosol particles, their density and other physical properties as well as parameters for the composition of the aerosol from different substances.

By using a larger number of deposition parameters, the accuracy of the deposition determination and thus ultimately the accuracy of the deposition result can be increased.

The deposition parameters can be entered and/or stored in the analysis device 105 or in the assessment device 160. In order to enable the greatest possible protection of patient data, patient-specific deposition parameters can be entered and stored only locally. It is also possible to transmit the patient-specific deposition parameters in encrypted form. In contrast, aerosol-specific deposition parameters can be retrieved from the respective manufacturer's servers, depending on the application, in order to enable rapid commissioning and error-free assessment.

It is also possible for stored deposition parameters to be linked to a specific serial number. A simplified deposition determination is then made possible by transmitting only the serial number to the assessment device, whereby the deposition parameters are retrieved from the assessment device using the serial number. This enables an even simpler and more secure assessment, since in the event of a potential unauthorized query of the serial number, it is at least not possible to directly infer sensitive patient-specific data—the stored deposition parameters.

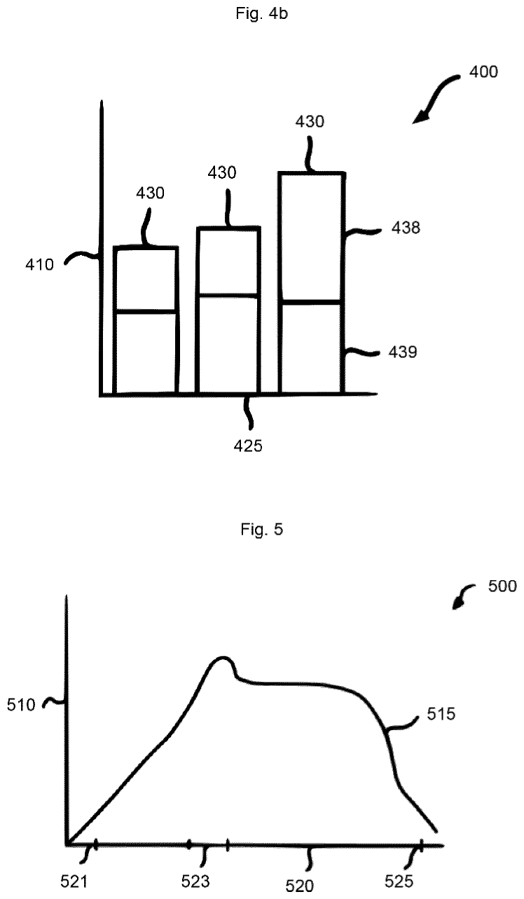

FIG. 4a shows a preferred deposition result 400. The deposition result 400 is a representation of a two-dimensional diagram. The first dimension is represented by a vertically arranged axis 410. The second dimension is represented by a horizontally arranged aerosol diameter axis 420. Seven bars 430 are shown along the aerosol diameter axis 420. The bars 430 are located at different positions along the aerosol diameter axis 420 and thus correspond to different aerosol diameters. The bars 430 each consist of an exhaled fraction 432, a lung fraction 434 and a throat fraction 436. The three fractions 432, 434, 436 are arranged one above the other and thus cumulatively correspond to a total fraction, for example a fraction of 100%. All seven bars 430 shown have the same total proportion, although the individual fractions 432, 434, 436 may differ. Using the deposition result 400 shown in FIG. 4a, conclusions can be drawn about the different distribution of aerosols in the lungs and throat and about the exhaled, i.e. exhaled, fraction 432 as a function of different aerosol diameters.

FIG. 4b shows a further preferred deposition result 400. The deposition result 400 is a representation of a two-dimensional diagram. The first dimension is represented by a vertically arranged axis 410. The second dimension is represented by a horizontally arranged inhalation duration axis 425. Three bars 430 are shown along the inhalation duration axis 425. The bars 430 are located at different positions of the inhalation duration axis 425 and thus correspond to different inhalation durations. The bars 430 each consist of a peripheral lung fraction 438 and a central lung fraction 439. The two fractions 438, 439 are arranged one above the other and thus cumulatively correspond to a total fraction. The three bars 430 shown each have a different total fraction and different peripheral lung fractions 438 and central lung fractions 439. Using the deposition result 400 shown in FIG. 4b, conclusions can be drawn about the different distribution of aerosols in the lungs, in particular in their peripheral and central areas, depending on different inhalation durations.

Further deposition results 400 in the form of diagrams are possible, for example a bar diagram in which each inhalation train is shown as a separate bar, whereby the individual bars corresponding to an inhalation train are arranged next to each other along a time axis in chronologically descending or ascending order. Since an inhalation or therapy session may well comprise 1,000 inhalation puffs, depending on the apparatus used and the amount of medication, it is appropriate in such a case for reasons of clarity to output a summarizing or cumulative bar chart as an additional deposition result 400. This cumulative deposition result 400 can also be output as a line diagram.

Other deposition results 400 are also possible, for example inhalation assessments or inhalation scores, which are better or higher the better the user has adhered to targets during inhalation or the better the user has achieved these targets.

Furthermore, feedback can be given to the user for subsequent applications or set on the ventilator or additional device for controlled inhalation as to how better or optimal deposition of the drug aerosols can be achieved. This can be done, for example, by providing the user with breathing patterns with a sequence of inhalation and exhalation sequences of different lengths and intensities. In this way, the user suffering from bronchitis can be given breathing patterns that lead to optimal deposition of the drug aerosols in the bronchi.

The deposition model can also be extended in such a way that the absorption of the active ingredient or active ingredients in the body is calculated based on the deposition result 400.

The control unit 130 can also be set up to control the first air sensor 120 in such a way that a large number of successive partial measurement results can be measured. In this way, the patient can receive continuous feedback during an inhalation as to the extent to which the current deposition result 400 corresponds to any target specifications. If necessary, the patient can adjust his respiratory flow in order to achieve a better deposition result 400, i.e. to deliver the aerosol 220 to the desired position to the desired extent.

FIG. 5 shows a time course of a first air flow parameter 515 as a preferred first measurement result 500. In the embodiment shown, the first air flow parameter 515 or the first measurement result 500 was measured in the course of nebulization of a medication aerosol by means of a nebulizer. The first measurement result 500 is a representation of a two-dimensional diagram. The first dimension is represented by a vertically arranged air flow parameter axis 510. The second dimension is represented by a horizontally arranged time axis 520. Three events 521, 523, 525 are shown along the time axis 520. The three events 521, 523, 525 are located at different positions of the time axis 520. At the beginning of the time axis 520 is the event 521, at which the nebulization was started. From this event 521, the first air flow parameter 515 increases. Along the time axis 520 to the right follows the event 523, which corresponds to the time period at which nebulization was paused. At this event 523, the first air flow parameter 515 reaches its maximum value and then decreases again. This is followed at the right end of the time axis by event 525, at which the nebulization was ended. At this event 525, the first air flow parameter 515 tends towards zero again. With the aid of the first measurement result 500, at least one deposition parameter and a deposition model, a deposition determination can then be carried out.

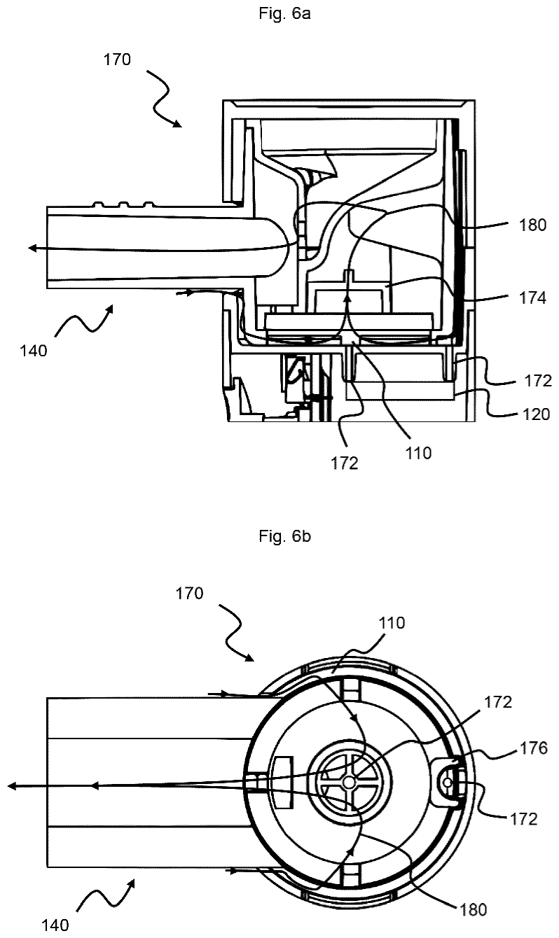

FIG. 6a shows a head unit 170 with application component 140 in a side sectional view with the air flow 180 drawn in. The air flow 180 enters the lower area of the head unit 170 at the side of the application component 140 by means of a bypass principle. In the embodiment shown, the air duct 110 is arranged below the head unit 170. The air flow 180 is guided in the air duct 110 along the first air sensor 120, the latter being connected to the air duct 110 via two sensor feed 172. After the air flow 180 has flowed along the first air sensor 120, it enters the head unit 170 centrally at the top via an outlet valve 174. The air flow 180 then exits to the outside via the application component 140 on the left-hand side.

FIG. 6b shows the head unit 170 shown in FIG. 6a in a sectional view from below. The air flow 180 enters the head unit 170 via the outer sides of the head unit 170 and is then bundled in the middle and directed outwards via the application component 140. The sensor feed 172 arranged on the right-hand side is shielded by an aperture 176. The centrally arranged sensor feed 172 is arranged within the air flow 180, while the sensor feed 172 arranged on the right is shielded from the air flow 180 by the aperture 176. This makes it possible to measure the differential pressure, which results on the one hand from a pressure influenced by the air flow 180—sensor feed 172 arranged in the middle—and on the other hand from a pressure not directly influenced by the air flow 180—sensor feed 172 arranged on the right. The differential pressure determined in this way can be used to determine the inhalation flow.

In addition to the measurement shown in FIGS. 6a and 6b, there are other conceivable measuring principles, including thermal air mass measurement using, for example, a hot-wire anemometer, possibly designed as a bypass, arranged directly in the main flow; differential pressure measurement using two absolute pressure sensors between two measuring points in the main flow, in particular with flow resistance between the two measuring points; the differential pressure measurement via two absolute pressure sensors, whereby one absolute pressure sensor is arranged in the air duct 110 and a second measures against the atmosphere; and the differential pressure measurement by means of a single absolute pressure sensor arranged in the flow channel, whereby the change in atmospheric pressure is neglected. Other possible flow measurement principles are the mechanical volume meter, the variable area and differential pressure measurement, and ultrasonic, Coriolis, magnetic-inductive and thermal flow meters.

### LIST OF REFERENCE NUMBERS

100 Analysis system
105 Analysis device
110 Air duct
120 First air sensor
122 Second air sensor
130 Control unit
140 Application component
150 Inhaler
160 Assessment device
170 Head unit
172 Sensor feed
174 Outlet valve
176 Aperture
180 Air flow
210 Air
220 Aerosol
230 Breathing apparatus
300 Method
310 Step
320 Step
330 Step
340 Step
350 Step
400 Deposition result
410 Axis
420 Aerosol diameter axis
425 Inhalation duration axis
430 Bar
432 Exhaled fraction
434 Lung fraction
436 Throat fraction
438 Peripheral lung fraction
439 Central lung fraction
500 First measurement result
510 Air flow parameter axis
515 First air flow parameter
520 Time axis
521 Event
523 Event
525 Event

1. Analysis system for determining the deposition of an aerosol for a breathing apparatus with an analysis device having an air duct, a first air sensor, a control unit and an application component, wherein
the air duct is set up to guide air along the first air sensor to the application component and vice versa,
the first air sensor is set up to detect at least a first air flow parameter of the air guided along the first air sensor, and
the control unit is set up to control the first air sensor in such a way that a time characteristic of the first air flow parameter can be measured as a first measurement result, and the first measurement result can be transmitted to an assessment device.

2. The analysis system according to claim 1, wherein the analysis system has the assessment device, and wherein the assessment device is set up to carry out a deposition determination using the first measurement result, at least one deposition parameter, and a deposition model, and to output a deposition result as the result of the deposition determination.

3. The analysis system according to claim 2, wherein the control unit is furthermore set up to store the first measurement result, and the first measurement result can be transmitted from the control unit to the assessment device.

4. The analysis system according to claim 1, wherein the control unit is further arranged to control the first air sensor such that a plurality of successive partial measurement results are measurable.

5. The analysis system according to claim 1, wherein the Analysis system further comprises a second air sensor which is arranged in the air duct, and wherein the control unit is further arranged to control the second air sensor such that a time course of the second air flow parameter can be measured as a second measurement result.

6. The analysis system according to claim 1, wherein the first air sensor is further arranged to detect a second air flow parameter of the air guided along the first air sensor, and wherein the control unit is further arranged to control the first air sensor such that a time course of the second air flow parameter can be measured as a second measurement result.

7. The analysis system according to claim 1, further comprising an inhaler, wherein the inhaler is in particular a nebulizer, particularly preferably a mesh nebulizer.

8. A method for determining the deposition of an aerosol for a breathing apparatus comprising the steps of:
i. Directing air in an air duct along a first air sensor to an application component;
ii. Detecting at least a first air flow parameter of the air guided along the first air sensor by means of the first air sensor;
iii. Controlling the first air sensor by means of a control unit in such a way that a time characteristic of the first air flow parameter is measured as a first measurement result;
iv. Performing a deposition determination using the first measurement result, at least one deposition parameter and a deposition model; and
v. Outputting a deposition result as a result of the deposition determination performed during step iv.

9. The method according to claim 8, wherein between steps iii. and iv. the first measurement result is received by means of an assessment device and step iv. is carried out by means of the assessment device.

10. The method according to claim 9, wherein during step iii. the first measurement result is stored in the control unit after the measurement and is then transmitted from the control unit to the assessment device.

11. The method according to claim 8, wherein during step iii. the first air sensor is controlled by means of the control unit such that a plurality of successive partial measurement results are measured.

12. The method according to claim 11, wherein during step iv. a deposition determination is carried out at a plurality of successive points in time in each case with the partial measurement results measured up to the current point in time and during step v. a current deposition result is output at the plurality of successive points in time in each case with the partial measurement results measured up to the current point in time during step iii.

13. The method according to claim 8, wherein during step i. further air is guided along a second air sensor (122) to the application component, during step ii. further a second air flow parameter of the air guided along the second air sensor is detected by means of the second air sensor, during step iii. furthermore, the second air sensor is controlled by means of the control unit in such a way that a time course of the second air flow parameter is measured as a second measurement result, and while step iv. the deposition determination is additionally carried out on the basis of the second measurement result.

14. The method according to claim 8, wherein during step ii. furthermore a second air flow parameter of the air guided along the first air sensor is detected by means of the first air sensor, while step iii. furthermore the first air sensor is controlled by means of the control unit such that a time course of the second air flow parameter is measured as a second measurement result, and while step iv. the deposition determination is additionally carried out on the basis of the second measurement result.

15. (canceled)
